(19) United States
(12) Patent Application Publication
(10) Pub. No.: US 2012/0083653 A1
(43) Pub. Date: Apr. 5, 2012

(54) GUIDED PROCEDURAL TREATMENT DEVICE AND METHOD

(76) Inventor: DANIEL P. SPERLING, West Orange, NJ (US)

(21) Appl. No.: 13/253,041

(22) Filed: Oct. 4, 2011

Related U.S. Application Data
(60) Provisional application No. 61/389,668, filed on Oct. 4, 2010.

(51) Int. Cl. A61B 1/00

(52) U.S. Cl. 600/104

(57) ABSTRACT

A medical device for minimally invasive surgical procedures. The medical device includes an ablative probe for removing malignant tissues from a body. A guidance sleeve encapsulates the ablative probe and facilitates its advancement through an opening in the body. In addition, the device includes an imaging device for monitoring the position of the guidance sleeve and the ablative probe within the body.

### CROSS-REFERENCE TO RELATED APPLICATIONS

This application claims the benefit of U.S. Provisional Patent Application No. 61/389,668, filed Oct. 4, 2010, which application is hereby incorporated by reference in its entirety.

### TECHNICAL FIELD

The present disclosure relates to minimally invasive surgical procedures, and more particularly to systems and method for improving the navigation of a medical device within a patient during minimally invasive surgical procedures.

### BACKGROUND

Prostate cancer is one of the most common types of cancer affecting men. It is a slow growing cancer, which is easily treatable if identified at an early stage. Treatment options for prostate cancer are primarily surgery, radiation therapy, chemotherapy, hormone therapy, and proton therapy.

Surgical methods, such as prostatectomy are highly invasive which require the patient to be hospitalized for many weeks. These methods might involve further complications during recovery. In light of these complications, physicians have recently turned to less invasive surgeries.

Less invasive procedures are typically called minimally invasive procedures or minimal incision techniques. It utilizes various targeted probes, such as thermal ablation probes (laser ablation, high-intensity focused ultrasound ablation, radio frequency ablation, etc.) and cryotherapy probes to reach the cancerous region, without cutting open the patient. Typically, these probes have provisions at their tips to deliver energy (in case of thermal ablation) or cryofluids (in case of cryotherapy probes) to the affected area. The probes are thin and flexible in order to reach sequestered regions with ease.

As the probes are inserted into a patient through small cuts or openings, it is difficult to position the probe correctly. To make this positioning easier and more accurate, external guidance systems are typically utilized to view the probe as it navigates through the patient's body to the affected area. A number of commonly known imaging systems may be utilized, such as MRI (magnetic resonance imaging), radiographic techniques (X-Ray, CT Scan), or ultrasonic imaging. Moreover, physicians might use specialized software, which may be customized for specific procedures to improve accuracy.

Currently available minimally invasive methods to treat prostate cancer involve transrectal procedures using any of the targeted probes described previously. The probes may be guided manually toward the cancerous region to remove the malignant parts of the prostate using any of the known image-guided systems. The probe, however, cannot independently advance through tissues, and it requires a guide to be employed in an existing cavity or vessel. Moreover, probes may face difficulty navigating to the desired region and may strike against rectal walls. For penetration through tissue layers and for effectively navigating the probe, a more stable guidance system is required. Consequently, there exists a need for a manual probe guiding device to ease the penetration, guidance and positioning of the probe through the transrectal wall of the colon to the prostate gland.

### SUMMARY

One embodiment of the present disclosure describes a medical device. The medical device includes an ablative probe for removing malignant tissues from a body. A guidance sleeve encapsulates the ablative probe for guiding the probe through an opening in the body. In addition, the device includes an imaging device for monitoring the position of the guidance sleeve and the ablative probe within the body.

Another embodiment of the present disclosure describes a method for performing a surgical procedure at a target site of a body. The method includes advancing a medical device into a body lumen. The medical device includes an ablative probe for removing malignant tissues from the body, a guidance sleeve surrounding the ablative probe, and an imaging device for monitoring the position of the guidance sleeve and the ablative probe within the body. The method includes capturing an image of the medical device within the body, and guiding the medical device proximate the target site based on the captured image.

### BRIEF DESCRIPTION OF THE DRAWINGS

The drawing figures described below set out and illustrate a number of exemplary embodiments of the claimed invention. Throughout the drawings, like reference numerals refer to identical or functionally similar elements. The drawings are illustrative in nature and are not drawn to scale.

FIG. 1 is an exemplary embodiment of a probe guidance system.

FIG. 2 illustrates the probe guidance system, shown in FIG. 1, in use for a transrectal prostrate procedure.

FIG. 3 illustrates an embodiment of the probe guidance system coupled to an interventional device.

### DETAILED DESCRIPTION

The following detailed description is made with reference to the figures. Exemplary embodiments are described to illustrate the claimed invention, not to limit its scope, which is defined by the appended claims.

### Overview

A system and method for focally and selectively destroying tumor tissue at the site of ablation is disclosed. To this end, the present disclosure employs a probe guidance system that relies on a convergence of technologies, including accurate imaging to detect and target malignant tissue, using systems such as MRI, combined with an appropriate tissue removal system, such as ablation probes. The system can also employ software in planning and executing the surgery.

### Exemplary Operating Environment

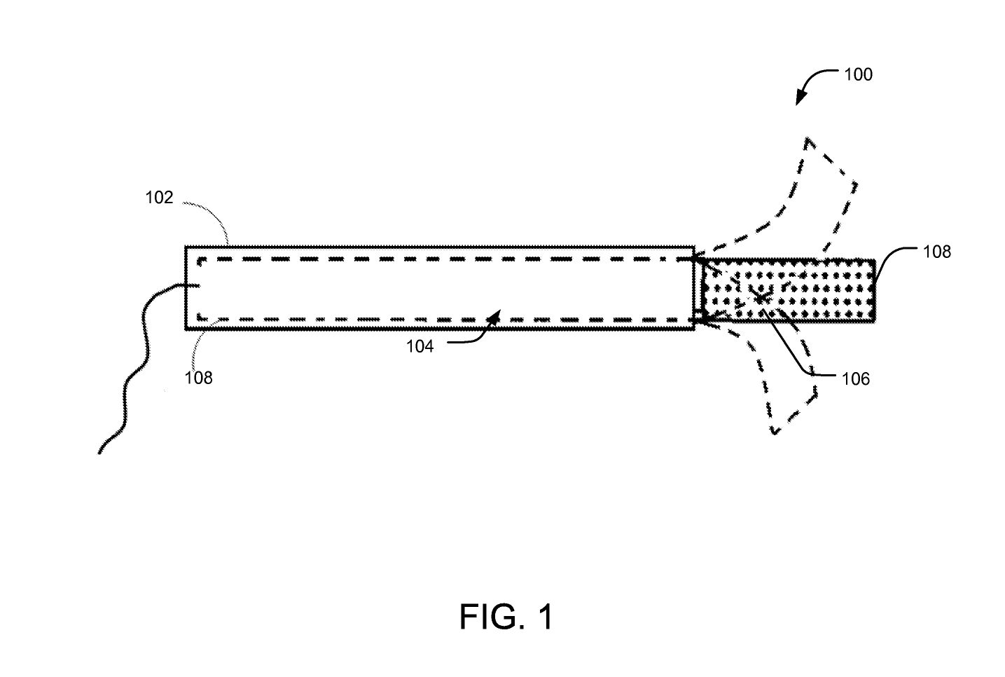

FIG. 1 illustrates an exemplary embodiment of a probe guidance system 100 for treating malignant tissues. The system includes a guidance sleeve 102 covering an ablation probe 104.

The guidance sleeve 102 may be a hollow cylindrical structure made of any suitable material, such as an MR compatible material. This sleeve 102 covers the ablative probe 104 (similar to needle sleeves used for biopsies), exposing the distal portion 106 of the probe 104 for easy navigation and application. The probe 104 may be slidably introduced into the sleeve 102 for performing the desired medical procedure. Once the procedure is complete, the probe 104 may be retracted.

The configuration and dimension of guidance sleeve 102 may vary, as desired. The illustrated embodiment depicts a uniform cylindrical structure; those skilled in the art, however, will appreciate that the sleeve 102 may be tapered along its edges or may include a non-uniform configuration based on the probe dimensions. The gauge size and the length of the guidance sleeve 102 can be customized according to the probe size. In one implementation, the guidance sleeve 102 may have a gauge in the range 10-18, and different guidance sleeves may be manufactured depending on the ablative probe dimensions. For example, the gauge of typical laser probes is 14 or lower, and a suitable corresponding sleeve can be manufactured to fit that sizing.

The ablation probe 104 may be an elongate tubular structure having a distal portion 106 and a proximal portion 108. The distal portion 106 of the probe 104 protrudes from the sleeve 102. Probe 104 may be made of a flexible, rigid, or semi-rigid material, as desired. In an embodiment, probe 104 may include a flexible outer housing extending along its length. The outer housing may be rigid enough to penetrate soft tissue, yet be flexible enough to follow curved or arcuate paths. Using any desired material for manufacture, the distal portion 106 may be flexible for convenient maneuver in a desired direction. To allow convenient insertion of the system 100 within a patient's body, a distal tip 110 of the probe 104 may be rigid having sharp ends. In an alternate embodiment, the probe 104 may lack the outer casing or the penetrating tip. That structure is suitable for a scenario in which the guidance sleeve 102 penetrates through soft tissue when required.

Once the probe 104 is deployed, the distal portion 106 may be bent towards the target tissue, focusing the ablation energy only toward the target tissue. The tip may be bent using any known steering mechanism such as pull wires. Alternatively, the distal portion 106 may be made of shape memory material that changes shape on exposure to a trigger, such as heat.

Moreover, any of the wide variety of ablative probes known in and available to the art can be employed using the guidance sleeve 102. For example, probes such as RF (radio frequency) probes, microwave probes, laser probes, or other known surgical probes may also be utilized. Other probes, known presently or later developed, may also be employed.

It may be desired to enhance the ability of surgical support systems to track the device during a procedure. To that end, guidance sleeve 102 may be adapted to operate in tandem with imaging systems known in the art, such as an ultrasound or radiographic system that assists the physician in navigating the guidance sleeve 102 and the probe 104 through a human/animal body.

In an embodiment of the present disclosure, the imaging system may be a 3-D imaging system that allows the MRI images to be electronically transferred and fused with the real-time ultrasound, providing a 3-D image similar to a roadmap to help guide the sleeve 102 into targeted areas accurately.

In addition, software, associated with a corresponding imaging device, may be customized for the planning and guidance of different procedures, such as a transrectal prostatectomy. Similarly, the software could be customized for localization and treatment of kidney or liver tumors or similar target tissue.

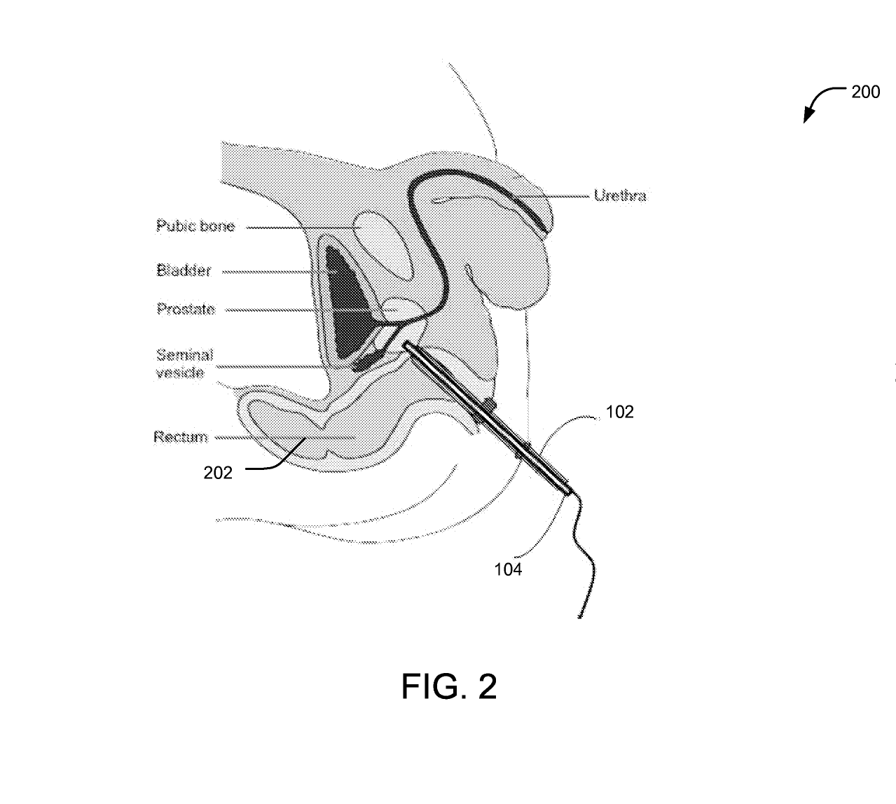

FIG. 2 illustrates the probe guidance system 100 in use for a transrectal prostrate procedure. For this procedure, the guidance sleeve 102 is first inserted into the rectum 202. MR images of the pelvic area are acquired in any suitable plane, such as the sagittal or axial plane, allowing visualization of the pelvic organs with the guidance sleeve 102 in place. Based on the images, software present in the MRI locks the position and the coordinates of this position may be applied to the guidance sleeve 102, accurately positioning it. The guidance sleeve 102 functions both as a guide for ablative probes and as a fiducial marker for the software.

Subsequently, any suitable ablative probe 104, such as a laser probe, may be safely inserted into the guidance sleeve 102. In an embodiment, the probe 104 extends through the length of the sleeve 102 such that the tip 110 of the probe 104 protrudes from the sleeve 102. Once positioned, the ablative probe 104 may be turned “on” to direct energy at the malignant tissue, destroying it.

The guidance sleeve 102 helps accurately position the ablative probe 104 into a patient's body to treat malignant tumors, such as cancers of the prostate, liver, etc. The ablative devices may include thermal ablation or cryotherapy probes. In addition to guiding the probes to the affected area, the guidance sleeve 102 also helps advance the probe 104 through tissue walls and other blockages to reach the affected area.

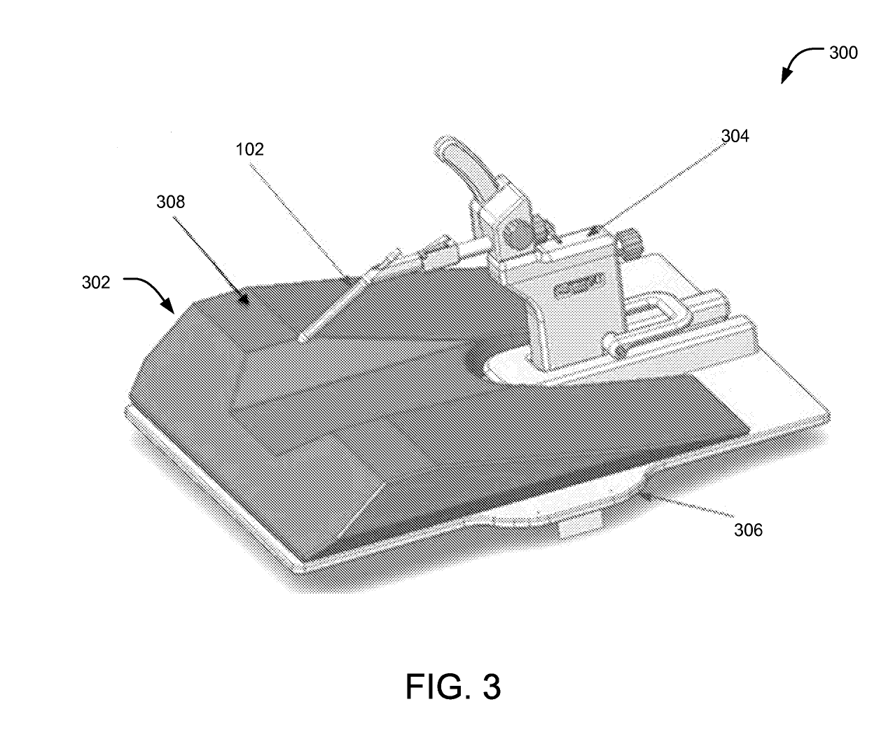

In an alternate embodiment 300 of the present disclosure, the guidance sleeve 102 may be attached to an interventional device 302 similar to the interventional device produced by Invivo® Corporation, depicted in FIG. 3. Here, the guidance sleeve 102 is mounted on a clamp stand 304, which is slidably positioned in a base plate 306. The clamp stand 304 can move in any axis, such as forward or backward and up or down, providing a high level of configurability. The interventional device 302 further includes a patient pad 308 for comfort. A patient may sit astride the interventional device 302; making is relatively easy for the guidance sleeve 102 to be inserted in the patient's rectum 202.

The medical device introduced here enables targeted destruction of radiographically visible tumor masses. Moreover, as the probe 104 is guided efficiently, the procedure becomes less painful (eliminating the need for anesthesia or sedation), and the guidance sleeve 102 minimizes complications.

The specification has set out a number of specific exemplary embodiments, but those skilled in the art will understand that variations in these embodiments will naturally occur in the course of embodying the subject matter of the disclosure in specific implementations and environments. It will further be understood that such variation and others as well, fall within the scope of the disclosure. Neither those possible variations nor the specific examples set above are set out to limit the scope of the disclosure. Rather, the scope of claimed invention is defined solely by the claims set out below.

I claim:

1. A medical device comprising:
an ablative probe for removing malignant tissues from a body;
a guidance sleeve surrounding the ablative probe for guiding the probe through an opening in the body; and
an imaging device for monitoring the position of the guidance sleeve and the ablative probe within the body.

2. The medical device of claim 1, wherein the distal portion of the ablation probe is flexible.

3. The medical device of claim 1, wherein the distal tip of the ablation probe is rigid.

4. The medical device of claim 1, wherein the guidance sleeve is an elongated hollow member.

5. The medical device of claim 1 further comprising an interventional device coupled to the guidance sleeve.

6. The medical device of claim 1, wherein the guidance sleeve includes fiducial markers.

7. A method for performing a surgical procedure at a target site of a body, the method comprising:
advancing a medical device into a body lumen, wherein the medical device includes:
an ablative probe for removing malignant tissues from the body;
a guidance sleeve surrounding the ablative probe; and
an imaging device for monitoring the position of the guidance sleeve and the ablative probe within the body;
capturing an image of the medical device within the body; and
positioning the medical device proximate the target site based on the image captured.

8. The method of claim 7 further comprising ablating the target site using the ablative probe.
